(19) United States
(12) Patent Application Publication
(10) Pub. No.: US 2024/0374892 A1
(43) Pub. Date: Nov. 14, 2024

(54) Directable Tunnel Device for Subcutaneous Implantable Cardio Defibrillator

(71) Applicant: Drexel University, Philadelphia, PA (US)

(72) Inventor: Randy Michael Stevens, Merion Station, PA (US)

(73) Assignee: Drexel University, Philadelphia, PA (US)

(21) Appl. No.: 18/289,599

(22) PCT Filed: May 6, 2022
(86) PCT No.: PCT/US22/27974

§ 371 Date: Nov. 6, 2023

Related U.S. Application Data
(60) Provisional application No. 63/186,867, filed on May 11, 2021.

(51) Int. Cl. A61N 1/05; A61B 17/32

(52) U.S. Cl. CPC A61N 1/0504; A61B 2017/320056

(57) ABSTRACT

A directional tunnel device for a subcutaneous implantable device is provided. The tunnel device includes an elongate outer sleeve having an open proximal end and an open distal end. An elongate outer trough has a trough proximal end, a trough distal end, and a concave cradle extending between the trough proximal end and the trough distal end. The outer trough is sized to slidingly fit inside outer sleeve such that the outer sleeve is slidable externally along the outer trough. An inner glide rests in the cradle. The inner glide includes an inner trough, a guide arm connected to a distal end of the inner trough, and a pivot arm connected to a distal end of the guide arm.

### CROSS-REFERENCE TO RELATED APPLICATION

The present application claims the benefit of U.S. Provisional Patent Application Ser. No. 63/186,867, filed on May 11, 2021, which is incorporated herein by reference in its entirety.

### BACKGROUND OF THE INVENTION

### Field of the Invention

The invention relates to a tunnel device that is used to redirect a medical device within subcutaneous tissue during insertion or implantation into a patient.

### Description of the Related Art

The insertion of subcutaneous implantable devices, such as cardio defibrillators, can be very difficult due to the tortuous path that the device must traverse in order to be placed at the desired location. The device must be redirected several times, some times turning as much as ninety degrees inside subcutaneous tissue. This process is especially difficult in pediatric and neonatal patients, whose smaller anatomy adds to the difficulty in performing the procedure.

It would be beneficial to provide a tunneler to assist in guiding subcutaneous implantable devices without having to perform an incision to turn the device to redirect it during insertion.

### BRIEF SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the present invention is a tunnel device for a subcutaneous implantable device. The tunnel device includes an elongate outer sleeve having an open proximal end and an open distal end. An elongate outer trough has a trough proximal end, a trough distal end, and a concave cradle extending between the trough proximal end and the trough distal end. The outer trough is sized to slidingly fit inside outer sleeve such that the outer sleeve is slidable externally along the outer trough. An inner glide rests in the cradle. The inner glide includes an inner trough, a guide arm connected to a distal end of the inner trough, and a pivot arm connected to a distal end of the guide arm.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a side elevational view of a tunnel device according to an exemplary embodiment of the present invention;

FIG. 1A is a side elevational view of a tunnel device according to an alternative exemplary embodiment of the present invention;

FIG. 2 is an exploded view of the tunnel device of FIG. 1;

FIG. 3 is a sectional view of a pivot arm of the tunnel device of FIG. 2, taken along lines 3-3 of FIG. 2;

FIG. 4 is a side elevational view showing an electrode being advanced using the tunnel device of FIG. 1;

FIG. 5 is a side elevational view of the tunnel device of FIG. 1, beginning deployment;

FIG. 6 is a side elevational view of the tunnel device of FIG. 5, in an advanced stage of deployment;

FIG. 7 is a side elevational view of the tunnel device of FIG. 5, with deployment completed;

FIG. 8 is a schematic view of the tunnel device of the present invention being used in a coronary procedure to obtain left atrial access;

FIG. 9 shows the tunnel device of FIG. 1 advanced to the bottom of the sternum of a patient;

FIG. 10 shows the device of FIG. 9 in a deployed position at the sternum;

FIG. 11 shows the device of FIG. 10 with the electrode separated from the device;

FIG. 12 shows the device moving to a retracted position;

FIG. 13 shows the device being withdrawn with the electrode in position; and

FIG. 14 shows the electrode in position.

### DETAILED DESCRIPTION OF THE INVENTION

In the drawings, like numerals indicate like elements throughout. Certain terminology is used herein for convenience only and is not to be taken as a limitation on the present invention. The terminology includes the words specifically mentioned, derivatives thereof and words of similar import. As used herein, the term“proximal” means a direction toward to the interventionist inserting the inventive tunneler and the term “distal” means a direction away from the interventionist inserting the inventive tunneler.

The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

As shown in the Figures, the present invention is a tunnel device (“device 100”) for implanting a defibrillator coil or electrode 180 of a subcutaneous implantable cardioverter-defibrillator (S-ICD) according to an exemplary embodiment of the present invention. Device 100 is used to turn electrode 180 about 90 degrees within the chest cavity of a patient, without having to perform an incision at the turning location.

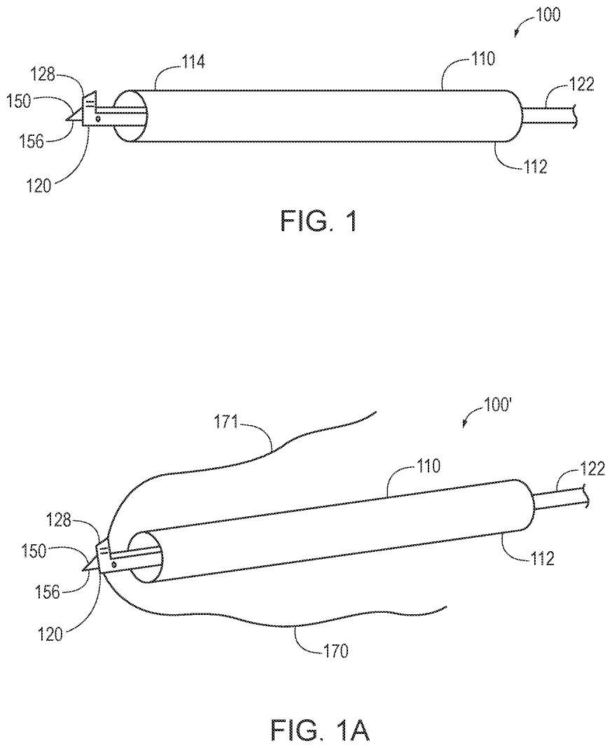

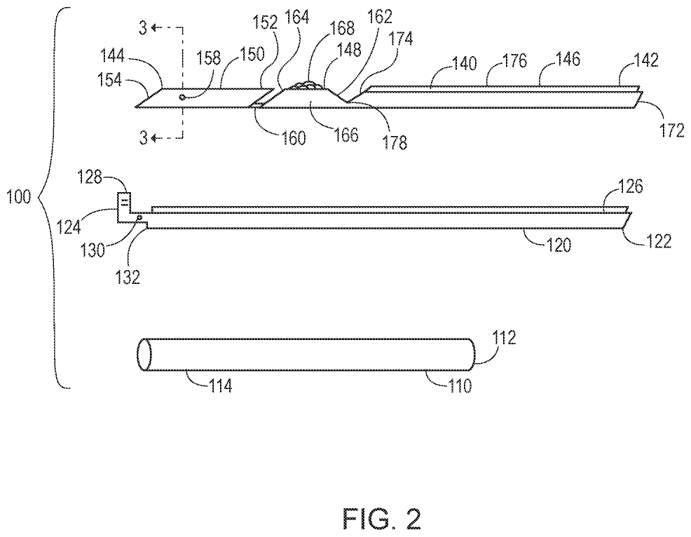

Referring to FIGS. 1 and 2, device 100 includes an elongate outer sleeve 110 having an open proximal end 112 and an open distal end 114. Outer sleeve 110 is generally tubular in shape and is used to protect the remaining portions of device 100 while device 100 is being advanced subcutaneously during implantation into a patient. Optionally, outer sleeve 110 can include a proximal handle (not shown) to allow an interventionalist to manipulate outer sleeve 110 during the insertion process.

An elongate outer trough 120 is sized to slidingly fit inside outer sleeve 110 so that outer sleeve 110 can be slid and directed externally along outer trough 120, such as in a distal direction, after device 100 is inserted at a desired location. Outer trough 120 has a proximal end 122, a distal end 124, and a concave cradle 126 extending between the proximal end 122 and the distal end 124. In an exemplary embodiment, cradle 126 has a generally semi-circular cross section and extends from proximal end 122 toward distal end 124, although those skilled in the art will recognize that cradle 126 can have other shapes. Cradle 126 is sized to allow inner glide 140 to rest in cradle 126. Further, proximal end 122 can have a generally annular cross section, if desired.

Distal end 124 includes a stop 128 that extends above cradle 126. Stop 128 acts as a positive brake to limit operation of an inner glide 140, as will be discussed below. A pivot 130 extends across trough 126 just proximally of stop 128. Additionally, the bottom surface of outer trough 120 proximal of stop 128 includes a longitudinally extending slot 132.

Inner glide 140 slidingly rests inside outer trough 120 and includes a proximal end 142 and a distal end 144. Proximal end 142 can be manipulated by the interventionist to longitudinally translate inner glide 140 along outer trough 120.

Inner glide 140 includes, from proximal end 142 to distal end 144, an inner trough 146, a guide arm 148 connected to a distal end of inner trough 146, and a pivot arm 150 connected to a distal end of guide arm 148.

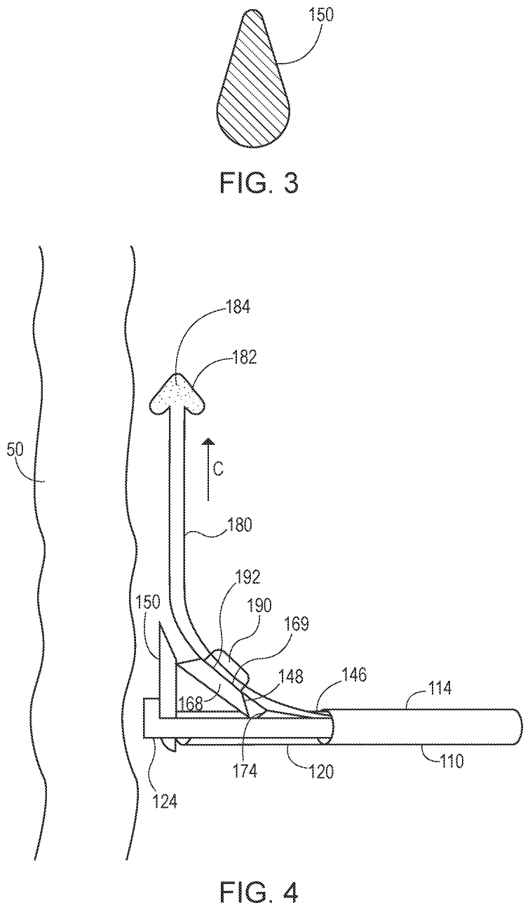

Pivot arm 150 includes a proximal end 152 and a distal end 154. Distal end 154 has a generally blunted tip 156 that can be used as a tunneler to dissect tissue as device 100 is being advanced subcutaneously. A pivot point 158 is located proximally of tip 156 and is pivotally connected to pivot 130 so that pivot arm 150 can pivot about pivot 130. Distal end 154 of pivot arm 150 can be reduced in lateral cross section such that distal end 154 of pivot arm 150 can fit into slot 132 when pivot arm 150 is pivoted. Optionally, pivot arm 150 can have a teardrop shaped cross section as shown in FIG. 3, with a generally blunt narrow length such that, when pivot arm 150 is pivoted during insertion of electrode 180, pivot arm 150 separates tissue to allow pivot arm 150 to fully pivot.

In an exemplary embodiment, pivot arm 150 is solid, although those skilled in the art will recognize that pivot arm 150 can include a cannula (not shown) to allow for the passage of fluids through pivot arm 150, if so desired.

Pivot arm 150 is connected to guide arm 148 via a living hinge 160. In an exemplary embodiment, as shown in FIG. 2, proximal end 152 of pivot arm 150 is tapered with a slope from top to bottom in a proximal-to-distal direction. Guide arm 148 has a proximal end 162 and a distal end 164, with a guide body 166 extending between the proximal end 162 and the distal end 164. The top surface of guide body 166 can include a first portion 168 of a releasable connector 169, such as a hook and loop fastener. The releasable connector 169 releasably connects electrode 180 to guide arm 148 such that, as pivot arm 150 is pivoting, electrode 180 stays adjacent to guide arm 148. After electrode 180 is deployed, releasable connector 169 releases electrode 180 from guide arm 148 so that pivot arm 150 can be rotated back to its insertion position so that device 100 can be withdrawn from the patient.

The distal end 164 is tapered with a slope from top to bottom in a proximal-to-distal direction to match the slope of proximal end 152 of pivot arm 150 such that, when inner glide 140 is being advanced with outer trough 120, distal end 164 of guide arm 148 extends under proximal end 152 of pivot arm 150. In an exemplary embodiment, the slope of tapered proximal end 152 of pivot arm 150 and tapered distal end 164 of guide arm 148 are both about 45 degrees.

Inner trough 146 includes a proximal end 172, a distal end 174, and a concave cradle 176 extending between the proximal end 172 and the distal end 174. In an exemplary embodiment, cradle 176 has a generally semi-circular cross section and extends from proximal end 172 to distal end 174, although those skilled in the art will recognize that cradle 176 can have other shapes. Cradle 176 is sized to allow electrode 180 to rest inside cradle 176 as device 100 is being inserted.

Proximal end 162 of guide arm 148 is connected to distal end 174 of cradle 176 via a living hinge 178. Proximal end 162 is tapered with a slope from top to bottom in a distal-to-proximal direction and distal end 174 of inner trough 146 is tapered with a slope from top to bottom in a proximal-to-distal direction such that a generally V-shaped gap is formed between proximal end 162 and distal end 174. In an exemplary embodiment, both proximal end 162 and distal end 174 are tapered about 22½ degrees.

Referring to FIG. 4, electrode 180 includes a barbed, generally flat distal tip 182, similar to an arrowhead in shape. This shape allows electrode 180 to be advanced generally parallel to the sternum, minimizing the likelihood that distal tip 182 veers away from the sternum as electrode 180 is advanced. Distal tip 182 includes an ingrowth surface 184 to encourage tissue ingrowth after deployment in order to stabilize the location of the distal tip 182 within the patient.

Electrode 180 also includes a sheath 190 surrounding electrode 180. Sheath 190 can be peeled away after electrode 180 is deployed. Sheath 190 also includes a second portion 192 of the releasable connector 169, such as a hook and loop fastener, that is releasably engaged with the first portion 168 of the releasably connector, in order to maintain electrode 180 with device 100 as device 100 is being deployed.

Optionally, as shown in FIG. 1A, a device 100′, similar to device 100, can include steering wires 170, 171 at the distal end of device 100′ to enable the interventionist to steer distal tip 156 to guide device 100′ as desired. By pulling on selected wires 170, 171, the distal tip 156 can be turned relative to the remainder of device 100′ to redirect device 100′ as device 100′ is being subcutaneously advanced. While wires 170, 171 are shown in FIG. 1A as being outside of sleeve 110, those skilled in the art will recognize that wires 170, 171 can be typically located inside of sleeve 110.

The device 100 allows a surgeon to utilize a single incision to place a subcutaneous coil for S-ICD without the need for additional incisions. The device 100 allows tunneling of any device (coil, tube, etc) in the subcutaneous portion and acute angle adjustments.

Prior to inserting device 100, a pre-operative chest X-ray is performed to determine that the patient is acceptable for S-ICD. The patient is marked in the left chest prepped and draped in standard surgical fashion and R2 pads. An incision is made subcutaneously in the inframammary crease and meticulous hemostasis is obtained. A dissection is carried down to the fascial plane, leaving the muscle intact.

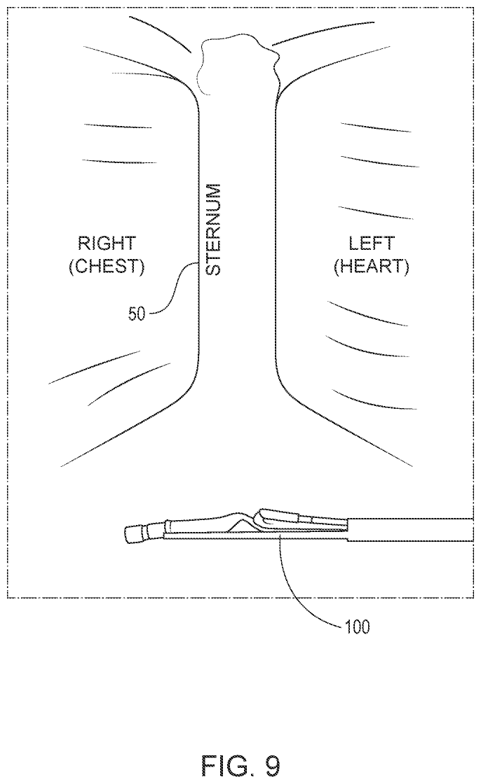

Next, device 100 is passed onto the sterile field and fluoroscopy can be used to ascertain the incision to the bottom of the sternum 50, as shown in FIG. 9. Device 100 is tunneled in the subcutaneous plane from the incision to the bottom of the sternum 50 (shown in FIG. 4) in a plane parallel to the chest wall.

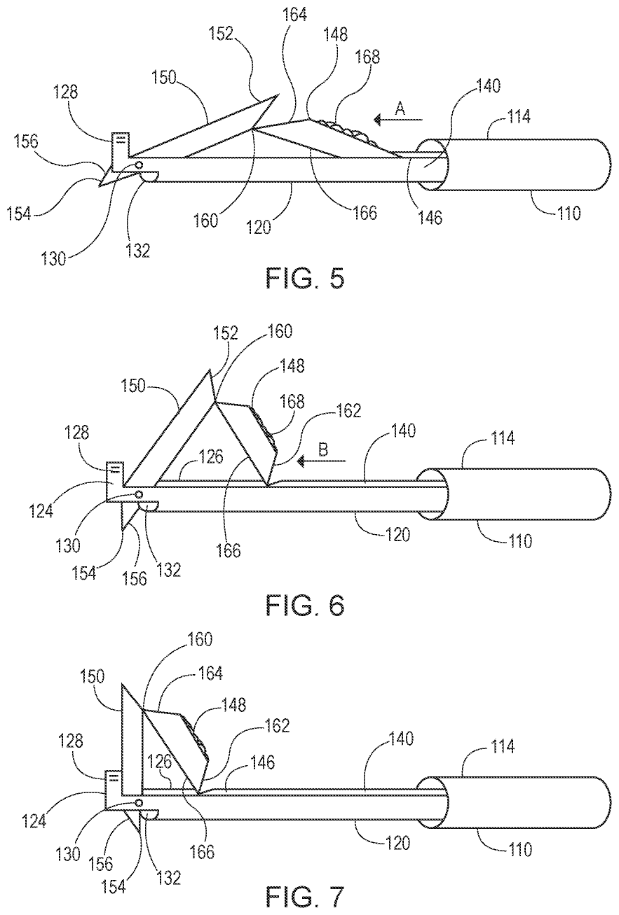

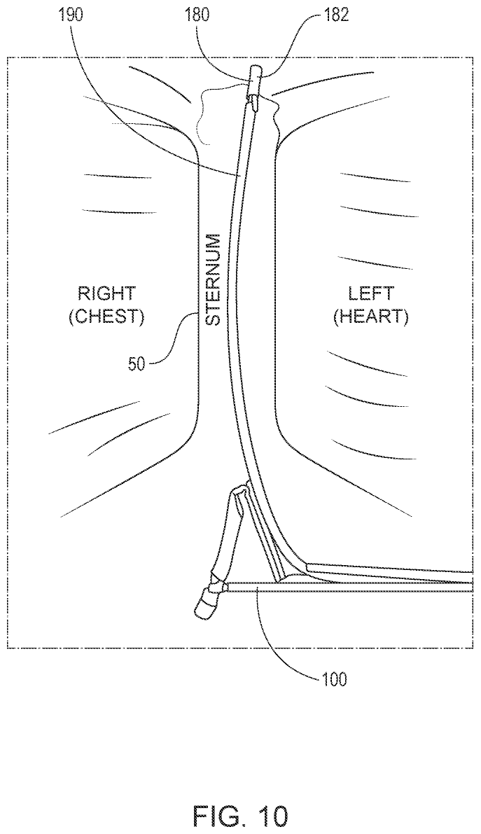

The device 100 is now deployed, creating the angle (90 degrees) as shown in FIG. 10. The inner glide 140 is advanced distally, as shown by arrow A in FIG. 5 and arrow B in FIG. 6. Pivot arm 150 pivots about pivot 130 such that distal tip 156 pivots into slot 132. Proximal end 152 pivots upwardly, bringing distal end 164 of guide arm 148 upwardly as well. Pivot arm 150 is pivoted until pivot arm 150 engages stop 128, as shown in FIG. 7. At this point, pivot arm 150 extends between about 85 degrees and about 90 degrees relative to the length of outer trough 120. Tapered proximal end 162 of guide arm 148 engages tapered distal end 174 of inner cradle 176 such that guide arm 148 is at an angle of about 45 degrees relative to the length of outer trough 120. Fluoroscopy is used to confirm a 90 degree angle.

Next the electrode 180 and inner sheath 190 (metal reinforced peel away) is advanced in a cephalad direction while using fluoroscopy. Electrode 180 is advanced distally along inner trough 146 until distal tip 182 engages guide arm 148. Guide arm 148 redirects distal tip 182 along guide arm 148 until electrode 180 is extending at an angle of about 90 degrees relative to the length of outer trough 120 and advancing upwardly along the spine to a desired location until the barbed tip 182 reaches the top of the xiphoid process of the sternum 50, as shown by arrow C in FIG. 4. Barbed tip 182 dissects tissue as electrode 180 is advanced. Gentle traction is used to pull on the coil confirming the barbed tip 182 has engaged in the subcutaneous tissue. The barbed tip 182 acts to anchor the electrode 180 at the top-most (cephalad) end of the tunnel. The barbed tip 182 consists of a plastic shape the helps dissect in a plane but is not sharp (not cutting). The tines or bottom ends can be overcome with enough pull so as to allow the electrode 180 to be completely removed in the event of infection.

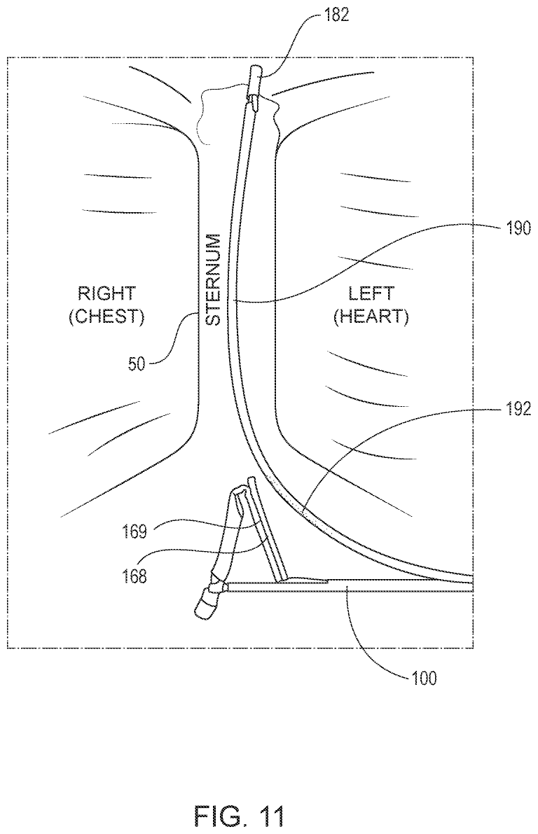

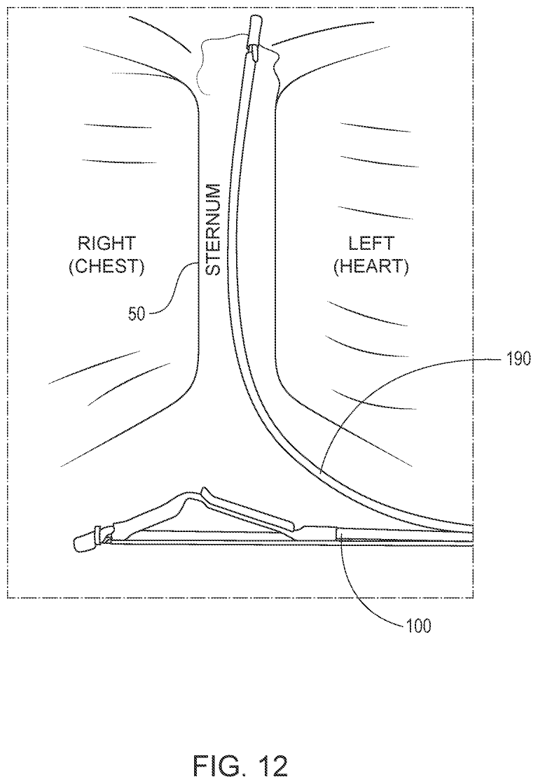

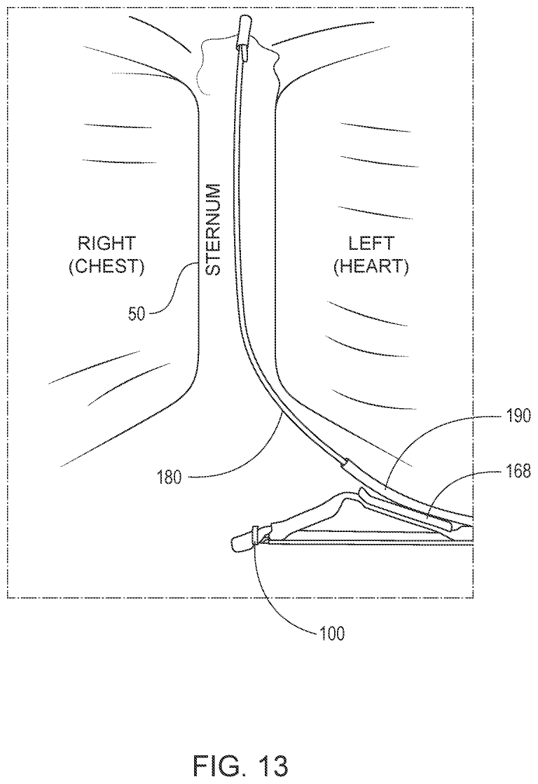

After electrode tip 182 has been advanced to a desired location, the interventionist retracts inner guide 140 proximally, thereby pivoting pivot arm 150 and guide arm 148 into outer trough 120. The first portion 168 of the releasable connector 169 releases from the second portion 192 of the releasable connector 169, thereby releasing electrode 180 from device 100, as shown in FIG. 11. Device 100 is then extracted proximally as shown in FIG. 12 and removed from the single incision. Under fluoroscopy and/or palpation the sheath 190 is removed and peeled away, leaving first portion 168 of releasable connector 169 as an anchor for electrode 180, as shown in FIG. 13.

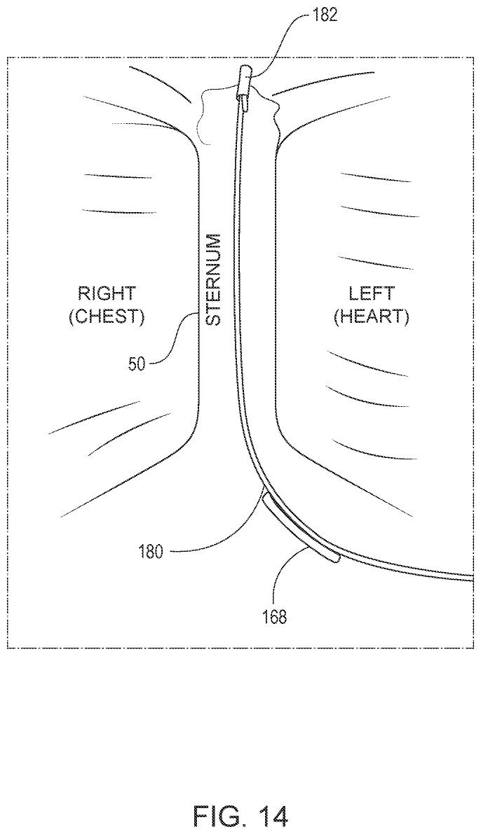

The living hinges 160, 178 on either side of guide body 166 have memory to favor a 90 degree angle of pivot arm 150 relative to outer trough 120 is maintained and has an outer covering of releasable connector 169 to favor engraftment of surrounding fascia and adipose tissue. With two points of fixation (barbed distal tip 182 and second part 192 of releasable connector 169) and deployment using fluoroscopy, the surgeon will have confidence that the electrode 180 will remain in position to complete the procedure as shown in FIG. 14.

The surgeon now connects the electrode or lead 180 to the ICD battery device and sutures the battery device to the fascia. The pocket is irrigated with antibiotic solution. The procedure is completed by suturing the fascia, dermis, and skin closed using appropriate suture. A sterile dressing is then applied.

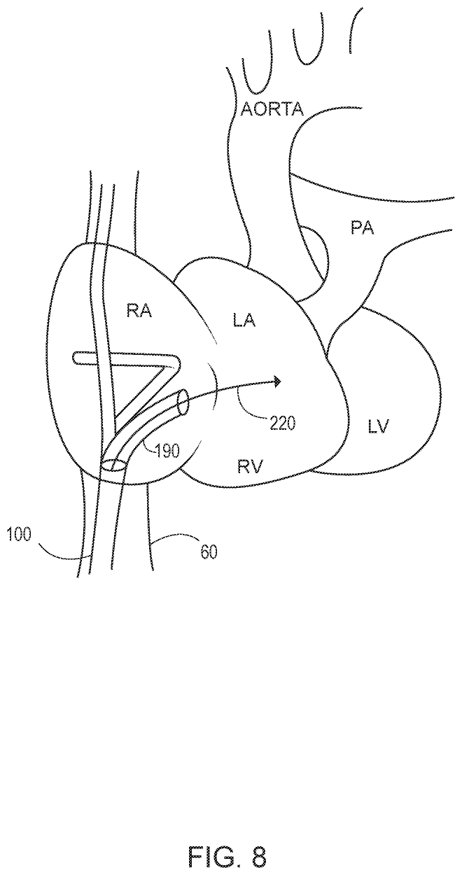

It is known that transeptal puncture is difficult. The device 100 can be used for angle creation of an intravascular device. An example of a transeptal puncture using device 100 is provided and shown schematically in FIG. 8, with the use of device 100 to insert electrode or lead 180 into a patient being shown in FIGS. 9-14.

First, needle access is provided in the groin. An introducer sheath is used. Under fluoroscopy and/or palpation, a guide wire 220 is advanced to the superior vena cava. The device 100 is advanced over the wire to the superior vena cava 60 and the guide wire 220 is removed. The inner guide 140 is advanced, while using fluoroscopy, to generate the angle needed. The outer sheath or introducer is not moved.

Next, the guide wire 220 and wire reinforced sheath are advanced, using fluoroscopy, to the septum of the heart. The guidewire 220 and wire reinforced sheath 190 are advanced until traversing the septum (from the right atrium RA to the left atrium LA). The outer trough 120 is advanced, removing the acute angle. The entire device 100 is removed by withdrawing the device 100 proximally along the guide wire 220, with the guide wire 220 and wire reinforced sheath 190 remaining in place. The wire reinforced sheath 190 is then removed and can be peeled away at the groin if needed.

The guidewire 220 at this point goes from venous access in the groin to the inferior vena cava 60 to the right atrium RA to the left atrium LA for “left heart access”. This procedure is suitable for mitral valve procedures, left atrial ablations, or any other indications.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A tunnel device for a subcutaneous implantable device, the tunnel device comprising:
an elongate outer sleeve having an open proximal end and an open distal end;
an elongate outer trough having a trough proximal end, a trough distal end, and a concave cradle extending between the trough proximal end and the trough distal end, the outer trough sized to slidingly fit inside outer sleeve such that the outer sleeve is slidable externally along the outer trough; and
an inner glide resting in the cradle, the inner glide including an inner trough, a guide arm connected to a distal end of the inner trough, and a pivot arm connected to a distal end of the guide arm.

2. The tunnel device according to claim 1, wherein the cradle has a generally semi-circular cross section.

3. The tunnel device according to claim 1, wherein the trough distal end comprises a stop extending above the cradle.

4. The tunnel device according to claim 3, wherein the trough comprises a pivot extending across the trough proximally of the stop.

5. The tunnel device according to claim 1, wherein the pivot arm includes a proximal end and a distal end.

6. The tunnel device according to claim 1, wherein the distal end has a generally blunted tip.

7. The tunnel device according to claim 6, further comprising a pivot point located proximally of the blunted tip and pivotally connected to the pivot such that the pivot arm is pivotable about the pivot.

8. The tunnel device according to claim 1, wherein the guide arm has a proximal end connected to a distal end of the inner trough via a living hinge.

9. The tunnel device according to claim 8, wherein the proximal end of the guide arm is tapered with a slope from top to bottom in a distal-to-proximal direction and the distal end of inner trough is tapered with a slope from top to bottom in a proximal-to-distal direction such that a generally V-shaped gap is formed between the proximal end of the guide arm and the distal end of the trough.

10. The tunnel device according to claim 1, wherein the pivot arm has a teardrop shaped cross section having a generally blunt narrow length such that when the pivot arm is pivoted during operation of the device, the pivot arm separates tissue to allow the pivot arm to fully pivot.

11. The tunnel device according to claim 1, wherein a proximal end of the pivot arm is tapered with a slope from top to bottom in a proximal-to-distal direction.

12. The tunnel device according to claim 11, wherein the slope of the tapered proximal end of the pivot arm is about 45 degrees.

13. The tunnel device according to claim 1, wherein the pivot arm is connected to the guide arm via a living hinge.

14. The tunnel device according to claim 1, further comprising a steering wire connected to the trough distal end.

15. The tunnel device according to claim 1 wherein a top surface of the guide arm includes a first portion of a releasable connector.

16. The tunnel device according to claim 15, further comprising a tool inserted into the inner trough.

17. The tunnel device according to claim 16, wherein the tool comprises an electrode.

18. The tunnel device according to claim 16, further comprising a sheath surrounding the tool.

19. The tunnel device according to claim 18, further comprising a second portion of the releasable connector attached to the sheath, such that the second portion of the releasable connector is releasably engaged with the first portion of the releasable connector.
